(19) United States
(12) Patent Application Publication
(10) Pub. No.: US 2025/0388722 A1
(43) Pub. Date: Dec. 25, 2025

(54) VIOLOGEN-BASEDIONIC POLYMER BINDER, AND PREPARATION METHOD AND USE THEREOF

(71) Applicant: NANKAI UNIVERSITY, Tianjin (CN)

(72) Inventors: Haixia Wei, Tianjin (CN); Wangqing Zhang, Tianjin (CN); Yuanyuan Xu, Tianjin (CN)

(21) Appl. No.: 18/905,598

(22) Filed: Oct. 3, 2024

(30) Foreign Application Priority Data
Jun. 24, 2024 (CN) 202410827739.3

(51) Int. Cl. C08G 65/00; C07D 401/04; H01M 4/02; H01M 4/04; H01M 4/62

(52) U.S. Cl. CPC C08G 65/00; C07D 401/04; H01M 4/0404; H01M 4/622; C08G 2650/22; H01M 2004/027; H01M 2004/028

(57) ABSTRACT

A viologen-based ionic polymer binder, and a preparation method and use thereof are provided, belonging to the technical field of lithium-ion batteries. The viologen-based ionic polymer binder is a polymer prepared from polymerization of a viologen-based acrylate, at least one of an acrylate and poly(ethylene glycol) methyl ether acrylate. When the viologen-based ionic polymer binder is used to prepare a cathode of a lithium-ion battery, a chain segment of the viologen-based polyacrylate could enhance bonding between cathode active materials, promote transportation of lithium ions, and thereby could improve capacity retention and recycling stability of the battery.

### CROSS REFERENCE TO RELATED APPLICATION

This patent application claims the benefit and priority of Chinese Patent Application No 2024108277393 entitled “Viologen-based ionic polymer binder, and preparation method and use thereof” filed on Jun. 24, 2024, the disclosure of which is incorporated by reference herein in its entirety as part of the present application.

### TECHNICAL FIELD

The present disclosure relates to the technical field of lithium-ion batteries, and in particular to a viologen-based ionic polymer binder, and a preparation method and use thereof.

### BACKGROUND

In cathodes of lithium-ion batteries, a binder is to bond the active materials and conductive additives onto the current collector to form stable cathodes and then maintain integrity of the cathodes during charging and discharging process. The binder shows a significant impact on the battery performances. When the binder exhibits poor adhesion, the active materials and conductive additives may fall off, causing battery failure; when the binder exhibits poor electrochemical stability, the binder may undergo irreversible chemical reactions with lithium ions, resulting in a decrease in battery capacity. Therefore, it is highly needed to design high-performance binders to solve problems such as active materials shedding from current collector and low recycling performance.

At present, the common binder for cathodes of lithium-ion batteries is polyvinylidene fluoride (PVDF). However, the PVDF exhibits several defects. For example, the interaction between PVDF and active materials through van der Waals forces is weak, which leads to poor adhesion of active materials and conductive additives onto the current collector. Besides, PVDF is nonconductive for electron/lithium ion, which is not helpful for Li⁺ transportation and therefore leads to high polarization especially at high charge/discharge current density. Furthermore, the interface impedance is relatively high when using PVDF as a cathode binder. In order to overcome these problems existing in PVDF binders, there have been many studies on cathode/anode binders for lithium-ion batteries. For example, CN111180733A discloses a binder containing ethylene carbonate, which endows the polymer binder with certain elasticity. However, the cathode prepared by this polymer binder still has poor adhesion, leading to capacity retention of the battery not more than 70%. For another example, CN111635478A discloses a low-impedance binder containing an ionic liquid structural unit. This binder shows excellent ion transportation. When the cathodes prepared by employing this binder are applied to a lithium-ion battery, the recycling performance of lithium-ion battery at low charge/discharge current density is improved. However, the lithium-ion battery has poor capacity retention and recycling stability at high charge/discharge current density.

As a result, it has become a technical challenge that needs to be solved urgently in the technical field of lithium-ion batteries to design polymer binders with enhanced adhesion performance and suitable electrochemical properties to keep high capacity retention and good recycling stability of lithium-ion batteries at high charge/discharge current density.

### SUMMARY

An object of the present disclosure is to provide a viologen-based ionic polymer binder, and a preparation method and use thereof. In the present disclosure, the viologen-based ionic polymer binder exhibits excellent capacity retention and recycling stability under high-rate charge and discharge conditions.

To achieve the above object, the present disclosure provides the following technical solutions:

The present disclosure provides a viologen-based ionic polymer binder, having a chemical structure shown in formula I.
formula I;

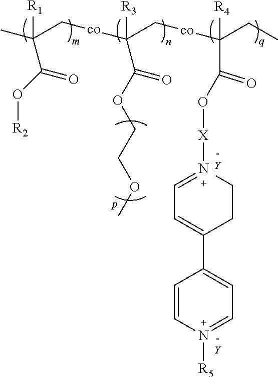

wherein in formula I, R₁, R₃, and R₄ are independently selected from the group consisting of hydrogen and methyl; R₂ and R₅ are independently alkyl; X is selected from the group consisting of methylene group, methylene ester group, and methylene carbamate group; Y is selected from the group consisting of PF₆, BF₄, Br, I, ½[SO₄], ½[CO₃], CH₃SO₃, CF₃SO₃, and (CF₃SO₂)₂N; m, n, and q satisfy m+n+q=1 where m>0, n>0, and q>0; and p is an integer in a range of 0 to 40. The present disclosure further provides a method for preparing the viologen-based ionic polymer binder as described in the above technical solutions, including the following steps: mixing a viologen-based acrylate, at least one of an acrylate and poly(ethylene glycol) methyl ether acrylate, an initiator, and a first solvent, and subjecting a resulting mixture to free radical polymerization, to obtain the viologen-based ionic polymer binder; wherein
the viologen-based acrylate has a chemical structure shown in formula II:
formula II, wherein

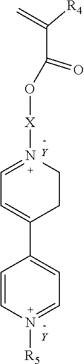

in formula II, R₄ is selected from the group consisting of hydrogen and methyl; R₅ is alkyl; X is selected from the group consisting of methylene group, methylene ester group, and methylene carbamate group; and Y is selected from the group consisting of PF₆, BF₄, Br, I, ½[SO₄], ½[CO₃], CH₃SO₃, CF₃SO₃, and (CF₃SO₂)₂N.

In some embodiments, the viologen-based acrylate is prepared by a process including the following steps:
(1) mixing 4,4′-bipyridine, an alkylating agent, and a second solvent, and subjecting a resulting mixture to a first alkylation reaction, to obtain an N-alkyl-4,4′-bipyridine salt;
(2) mixing the N-alkyl-4,4′-bipyridine salt obtained in step (1) with a 1-halogeno alkan-1′-ol and a third solvent, and subjecting a resulting mixture to a second alkylation reaction, to obtain a 1-(hydroxyalkyl)-1′-alkyl viologen salt (1);
(3) mixing the 1-(hydroxyalkyl)-1′-alkyl viologen salt (1) obtained in step (2), a salt containing an anion different from that of the 1-(hydroxyalkyl)-1′-alkyl viologen salt (1), and a fourth solvent, and subjecting a resulting mixture to ion exchange reaction, to obtain a 1-(hydroxyalkyl)-1′-alkyl viologen salt (2); and
(4) mixing the 1-(hydroxyalkyl)-1′-alkyl viologen salt (2) obtained in step (3) with a vinyl carbonyl compound, a catalyst, a polymerization inhibitor, and a fifth solvent, and subjecting a resulting mixture to esterification reaction, to obtain the viologen-based acrylate.

In some embodiments, in step (1), the alkylating agent is at least one selected from the group consisting of methyl iodide, dimethyl sulfate (DMS), dimethyl carbonate (DMC), ethyl iodide, diethyl sulfate (DES), diethyl carbonate (DEC), propyl iodide, dipropyl sulfate (DPS), and dipropyl carbonate (DPC); the second solvent is at least one selected from the group consisting of dichloroethane (DCE), tetrahydrofuran (THF), dichloromethane (DCM), toluene, and chloroform; and the first alkylation reaction is conducted at a temperature of 10° C. to 35° C. for 1 h to 24 h.

In some embodiments, in step (2), the 1-halogeno alkan-1′-ol is at least one selected from the group consisting of 2-bromoethanol, 2-chloroethanol, 3-bromo-1-propanol, 3-chloro-1-propanol, 4-bromo-1-butanol, 4-chloro-1-butanol, 5-bromo-1-pentanol, 5-chloro-1-pentanol, 6-bromo-1-hexanol, 6-chloro-1-hexanol, 7-bromo-1-heptanol, 7-chloro-1-heptanol, 8-bromo-1-octanol, and 8-chloro-1-octanol; the third solvent is at least one selected from the group consisting of acetonitrile, N-methylpyrrolidone (NMP), N,N-dimethylformamide (DMF), and dimethyl sulfoxide (DMSO); and the second alkylation reaction is conducted at a temperature of 50° C. to 100° C. for 30 h to 72 h.

In some embodiments, in step (3), the salt containing the anion different from that of the 1-(hydroxyalkyl)-1′-alkyl viologen salt (1) is at least one selected from the group consisting of KPF₆, NaPF₆, NH₄PF₆, NaBF₄, KBF₄, NaBr, KBr, NaI, KI, Na₂SO₄, Na₂CO₃, K₂CO₃, CH₃SO₃Na, CF₃SO₃Na, and (CF₃SO₂)₂NLi; the fourth solvent is at least one selected from the group of deionized water, ethanol, NMP, DMF, and DMSO; and the ion exchange reaction is conducted at a temperature of 10° C. to 35° C. for 1 h to 12 h.

In some embodiments, in step (4), the vinyl carbonyl compound is at least one selected from the group consisting of 2-isocyanatoethyl acrylate, isocyanatoethyl methacrylate, acrylic acid, methacrylic acid, acryloyl chloride, and methacryloyl chloride; the catalyst is at least one selected from the group consisting of dibutyltin dilaurate (DBTDL), triethylamine (TEA), sodium carbonate, and potassium carbonate; the polymerization inhibitor is at least one selected from the group consisting of p-methoxyphenol, p-benzoquinone, and p-hydroquinone; the fifth solvent is at least one selected from the group consisting of NMP, DMF, THF, 1,4-dioxane, acetonitrile, and chloroform; and the esterification reaction is conducted at a temperature of 10° C. to 35° C. for 1 h to 36 h.

In some embodiments, the initiator for the free radical polymerization is at least one selected from the group consisting of azobisisobutyronitrile (AIBN), 1,1′-azobis(cyclohexane-1-carbonitrile) (ACCN), dimethyl 2,2′-azobisisobutyrate, and dibenzoyl peroxide (BPO); the first solvent is at least one selected from the group consisting of acetonitrile, toluene, acetone, NMP, DMF, THF, 1,4-dioxane, and chloroform; and the free radical polymerization is conducted at a temperature of 50° C. to 80° C. for 8 h to 12 h.

The present disclosure further provides use of the viologen-based ionic polymer binder as described in the above technical solutions or the viologen-based ionic polymer binder prepared by the method as described in the above technical solutions in preparation of an electrode of a lithium-ion battery.

In some embodiments, the electrode of the lithium-ion battery is one selected from the group consisting of a cathode and an anode.

In the present disclosure, the viologen-based ionic polymer binder is a polymer prepared from a viologen-based acrylate, at least one of an acrylate and poly(ethylene glycol) methyl ether acrylate. When the binder is used to prepare a cathode of a lithium-ion battery, the chain segment of the viologen-based acrylate acts as follows: first, quaternary ammonium cations and counter anions in a viologen structure are combined with cathode active materials of the lithium-ion battery through electrostatic interaction, which could enhance the adhesion between the cathode active materials. Second, the counter anions in the viologen structure could promote the transportation of lithium ions. Third, the viologen has a stable structure and reversible redox properties. When the ionic polymer is used as a binder for cathode of a lithium-ion battery, it is beneficial to improve the capacity retention and recycling stability of the lithium-ion battery. The chain segment of the polyacrylate could not only improve the flexibility of the binder and soften the electrode, but also could enhance the hydrophobicity of the binder, thereby avoiding the decrease in battery capacity and recycling performance caused by the absorbed water. On the one hand, the chain segment of the poly(ethylene glycol) methyl ether acrylate could enhance the flexibility of the binder; on the other hand, due to desirable lithium ion transportation, the chain segment(s) could improve the ion transportation between the cathode and the electrolyte interface and reduce the interface impedance. Therefore, a lithium-ion battery assembled with the cathode prepared by the viologen-based ionic polymer binder according to the present disclosure has lower interface impedance, higher specific capacity, higher capacity retention, and desirable recycling stability. The results of examples show that the viologen-based ionic polymer binder has a glass transition temperature of −45.3° C. to −24.5° C. A cathode prepared with the viologen-based ionic polymer binder has a peel strength of 120.1-145.7 N·m⁻¹. A lithium iron phosphate|metal lithium battery assembled with the cathode prepared with the viologen-based ionic polymer binder has an initial discharge capacity of 141.5-145.2 mAh·g⁻¹ with a cut-off voltage of 2.5 V to 4.2 V and a rate of 0.5C. The battery after 400 cycles has a discharge specific capacity of 128.5-136.3 mAh·g⁻¹, showing a capacity retention of 89.17% to 93.87%. The lithium iron phosphate|metal lithium battery assembled with the cathode prepared from the viologen-based ionic polymer binder has an initial interface impedance of 204.1Ω to 211.9Ω.

### BRIEF DESCRIPTION OF THE DRAWINGS

FIG. 1 shows a hydrogen nuclear magnetic (¹H-NMR) spectrum of 1-(2-(((2-(acryloyloxy)ethyl)carbamoyl)oxy)ethyl)-1′-methylviologen hexafluorophosphate prepared in Example 1 of the present disclosure.

FIG. 2 shows an ¹H-NMR spectrum of the viologen-based ionic polymer binder A1 prepared in Example 1 of the present disclosure.

FIG. 3 shows an ¹H-NMR spectrum of 1-((2-acryloyloxy)ethyl)-1′-methylviologen hexafluorophosphate prepared in Example 3 of the present disclosure.

FIG. 4 shows an ¹H-NMR spectrum of the viologen-based ionic polymer binder A5 prepared in Example 5 of the present disclosure.

FIG. 5 shows a differential scanning calorimetry curve of the viologen-based ionic polymer binder A5 prepared in Example 5 of the present disclosure.

FIG. 6 shows the electrochemical impedance spectroscopy of lithium iron phosphatellithium metal batteries D5 and D10 prepared in the present disclosure at 25° C.

FIG. 7 shows long recycling charge-discharge curves of the lithium iron phosphatellithium metal batteries D5 and D10 prepared in the present disclosure at 25° C. with a cut-off voltage of 2.5 V to 4.2 V, and a rate of 0.5C.

FIG. 8 shows a charge-discharge curve of the lithium iron phosphatellithium metal battery D5 prepared in the present disclosure at 25° C. with a cut-off voltage of 2.5 V to 4.2 V and different rates of 0.2C, 0.5C, 1C, 2C, and 3C.

### DETAILED DESCRIPTION OF THE EMBODIMENTS

### Definition

In this text, m, n, and q separately represent mole fractions of corresponding repeating units in a polymer main chain with the proviso that a sum of m, n, and q is 100% (that is to say, m+n+q=1).

The present disclosure provides a viologen-based ionic polymer binder, having a chemical structure shown in formula I.

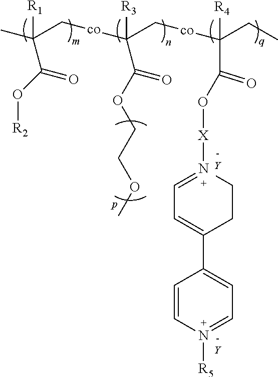

formula I,
wherein in formula I of the present disclosure, R₁, R₃, and R₄ are independently selected from the group consisting of hydrogen and methyl; R₂ and R₅ are independently alkyl; X is selected from the group consisting of methylene group, methylene ester group, and methylene carbamate group; Y is selected from the group consisting of PF₆, BF₄, Br, I, ½[SO₄], ½[CO₃], CH₃SO₃, CF₃SO₃, and (CF₃SO₂)₂NH; m, n, and q satisfy m+n+q=1 where m≥0, n≥0, and q>0; and p is an integer in a range of 0 to 40.

In some embodiments of the present disclosure, the alkyl is C₁ to C₄ alkyl, and preferably methyl, ethyl, or butyl. In some embodiments, R₂ is ethyl or butyl. In some embodiments, R₅ is methyl. p is an integer in a range of 0 to 40, preferably 9 to 30, and even more preferably 9, 10, 12, 15, 20, or 30. In some embodiments, m is in a range of 0 to 0.9, preferably 0, 0.62, 0.68, 0.81, 0.86, or 0.9. In some embodiments, n is in a range of 0 to 0.75, preferably 0, 0.05, 0.16, 0.65, or 0.75. In some embodiments, q is in a range of 0.10 to 1, preferably 0.1, 0.14, 0.16, 0.22, 0.25, 0.35, or 1. The viologen-based ionic polymer binder having a structure shown in formula I according to the present disclosure could not only make the binder have good adhesion but also promote the transportation of lithium ions, thereby improving the capacity retention and recycling stability of the battery.

The present disclosure further provides a method for preparing the viologen-based ionic polymer binder, including the following steps: mixing a viologen-based acrylate, at least one of an acrylate and poly(ethylene glycol) methyl ether acrylate, an initiator, and a first solvent, and subjecting a resulting mixture to free radical polymerization to obtain the viologen-based ionic polymer binder; wherein
the viologen-based acrylate has a chemical structure shown in formula II:

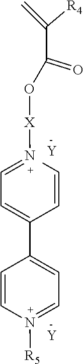

formula II, wherein
in formula II, R₄ is selected from the group consisting of hydrogen and methyl; R₅ is alkyl; X is selected from the group consisting of methylene group, methylene ester group, and methylene carbamate group; and Y is selected from the group consisting of PF₆, BF₄, Br, I, ½[SO₄], ½[CO₃], CH₃SO₃, CF₃SO₃, and (CF₃SO₂)₂NH.

In the present disclosure, unless otherwise specified, all materials used are commodities well known to those skilled in the art.

In some embodiments of the present disclosure, the viologen-based acrylate is prepared by a process including the following steps:
(1) mixing 4,4′-bipyridine, an alkylating agent, and a second solvent, and subjecting a resulting mixture to a first alkylation reaction, to obtain an N-alkyl-4,4′-bipyridine salt;
(2) mixing the N-alkyl-4,4′-bipyridine salt obtained in step (1) with a 1-halogeno alkan-1′-ol and a third solvent, and subjecting a resulting mixture to a second alkylation reaction, to obtain a 1-(hydroxyalkyl)-1′-alkyl viologen salt (1);
(3) mixing the 1-(hydroxyalkyl)-1′-alkyl viologen salt (1) obtained in step (2), a salt containing an anion different from that of the 1-(hydroxyalkyl)-1′-alkyl viologen salt (1), and a fourth solvent, and subjecting a resulting mixture to ion exchange reaction, to obtain a 1-(hydroxyalkyl)-1′-alkyl viologen salt (2); and
(4) mixing the 1-(hydroxyalkyl)-1′-alkyl viologen salt (2) obtained in step (3) with a vinyl carbonyl compound, a catalyst, a polymerization inhibitor, and a fifth solvent, and subjecting a resulting mixture to esterification reaction, to obtain the viologen-based acrylate.

In some embodiments of the present disclosure, 4,4′-bipyridine, an alkylating agent, and a second solvent are mixed, and a resulting mixture is subjected to a first alkylation reaction to obtain an N-alkyl-4,4′-bipyridine salt.

In some embodiments of the present disclosure, the alkylating agent includes at least one of methyl iodide, DMS, DMC, ethyl iodide, DES, DEC, propyl iodide, DPS, and DPC. In the present disclosure, the alkylating agent defined above undergoes alkylation reaction with the 4,4′-bipyridine to form the N-alkyl-4,4′-bipyridine salt.

In some embodiments of the present disclosure, the second solvent includes at least one of DCE, THF, DCM, toluene, and chloroform. In the present disclosure, the above solvent shows desirable solubility for the 4,4′-bipyridine and the alkylating agent, and thus is more conducive to fulfill the first alkylation reaction.

In some embodiments of the present disclosure, a mass ratio of 4,4′-bipyridine, the alkylating agent, and the second solvent is in a range of (160-250):(150-240):4000, preferably (190-240):(200-230):4000, and more preferably (195-235):(210-225):4000. The above mass ratio could avoid the formation of N,N-dialkyl-4,4′-bipyridine salt and is conducive to the formation of N-alkyl-4,4′-bipyridine salt.

In the present disclosure, there is no particular limitation on means for mixing 4,4′-bipyridine, the alkylating agent, and the second solvent, and any conventional mixing means may be used to fully dissolve the components. In some embodiments, 4,4′-bipyridine, the alkylating agent, and the second solvent are mixed under stirring.

In some embodiments of the present disclosure, the first alkylation reaction is conducted at a temperature of 10° C. to 35° C., preferably 15° C. to 30° C., and more preferably 20° C. to 30° C. In some embodiments, the first alkylation reaction is conducted for 1 h to 24 h, preferably 8 h to 20 h, and more preferably 10 h to 13 h. Controlling the temperature and time for the first alkylation reaction could avoid the formation of NN-dialkyl-4,4′-bipyridine salt and facilitate the formation of N-alkyl-4,4′-bipyridine salt.

In some embodiments of the present disclosure, a product obtained from the first alkylation reaction is mixed with a first precipitant, and a resulting mixture is subjected to filtration, followed by drying, to obtain the N-alkyl-4,4′-bipyridine salt.

In some embodiments of the present disclosure, the first precipitant is at least one of diethyl ether, n-hexane, and cyclohexane. In some embodiments, when the first precipitant is a mixture of two or more of the above, there is no special limitation on a ratio between the two or more, which may be mixed in any ratio. In the present disclosure, by using the first precipitant, the N-alkyl-4,4′-bipyridine salt could be separated from a reaction solution.

In some embodiments of the present disclosure, a mass ratio of the first precipitant to the product obtained from the first alkylation reaction is in a range of (4-11):1, and preferably (5-10):1. Controlling the amount of the first precipitant within the above range could fully precipitate the product obtained from the first alkylation reaction.

In the present disclosure, there is no particular limitation on operations of the filtering and drying, and operations well known to those skilled in the art may be used. In some embodiments, the drying is conducted at a temperature of 40° C. to 70° C., and preferably 50° C. to 60° C. In some embodiments, the drying is conducted for 8 h to 15 h, and preferably 10 h to 12 h.

In some embodiments of the present disclosure, after obtaining the N-alkyl-4,4′-bipyridine salt, the N-alkyl-4,4′-bipyridine salt is mixed with a 1-halogeno alkan-1′-ol and a third solvent, and a resulting mixture is subjected to a second alkylation reaction, to obtain a 1-(hydroxyalkyl)-1′-alkyl viologen salt (1).

In some embodiments of the present disclosure, the 1-halogeno alkan-1′-ol is at least one selected from the group consisting of 2-bromoethanol, 2-chloroethanol, 3-bromo-1-propanol, 3-chloro-1-propanol, 4-bromo-1-butanol, 4-chloro-1-butanol, 5-bromo-1-pentanol, 5-chloro-1-pentanol, 6-bromo-1-hexanol, 6-chloro-1-hexanol, 7-bromo-1-heptanol, 7-chloro-1-heptanol, 8-bromo-1-octanol, and 8-chloro-1-octanol. The above 1-halogeno alkan-1′-ol is selected to react with the N-alkyl-4,4′-bipyridine salt to form the 1-(hydroxyalkyl)-1′-alkyl viologen salt (1).

In some embodiments of the present disclosure, the third solvent is at least one selected from the group consisting of acetonitrile, NMP, DMF, and DMSO.

In some embodiments of the present disclosure, a mass ratio of the N-alkyl-4,4′-bipyridine salt, 1-halogeno alkan-1′-ol, and the third solvent is in a range of (390-420):(190-230):4000, and preferably (390-410):(195-230):4000.

In the present disclosure, there is no particular limitation on the mixing of the N-alkyl-4,4′-bipyridine salt, the 1-halogeno alkan-1′-ol, and the third solvent, and conventional mixing methods may be used to fully dissolve the above components.

In some embodiments of the present disclosure, the second alkylation reaction is conducted at a temperature of 50° C. to 100° C., and preferably 70° C. to 90° C. In some embodiments, the second alkylation reaction is conducted for 12 h to 72 h, preferably 40 h to 65 h, and more preferably 50 h to 63 h.

In some embodiments of the present disclosure, a product obtained from the second alkylation reaction is mixed with a second precipitant, and a resulting mixture is subjected to filtration, followed by drying, to obtain the 1-(hydroxyalkyl)-1′-alkyl viologen salt (1).

In some embodiments of the present disclosure, the second precipitant is at least one of DCM, DCE, diethyl ether, and n-hexane. In some embodiments, when the second precipitant is a mixture of two or more of the above, there is no special limitation on a ratio between the two or more, which may be mixed in any ratio.

In some embodiments of the present disclosure, a mass ratio of the second precipitant to the product obtained from the second alkylation reaction is in a range of (4-11):1, and preferably (6-11):1. In the present disclosure, the filtration and drying means and parameters thereof are the same as those of the filtration and drying after the product obtained from the first alkylation reaction is mixed with the first precipitant in the above technical solutions, and will not be described in detail here.

In some embodiments of the present disclosure, after obtaining the 1-(hydroxyalkyl)-1′-alkyl viologen salt (1), the 1-(hydroxyalkyl)-1′-alkyl viologen salt (1) is mixed with a salt containing an anion different from that of the 1-(hydroxyalkyl)-1′-alkyl viologen salt (1) and a fourth solvent, and a resulting mixture is subjected to ion exchange reaction to obtain a 1-(hydroxyalkyl)-1′-alkyl viologen salt (2). In some embodiments, the salt containing the anion different from that of the 1-(hydroxyalkyl)-1′-alkyl viologen salt (1) is at least one selected from the group consisting of KPF₆, NaPF₆, NH₄PF₆, NaBF₄, KBF₄, NaBr, KBr, NaI, KI, Na₂SO₄, Na₂CO₃, K₂CO₃, CH₃SO₃Na, CF₃SO₃Na, and (CF₃SO₂)₂NHLi. In some embodiments, when the salt containing the anion different from that of the 1-(hydroxyalkyl)-1′-alkyl viologen salt (1) is a mixture of two or more of the above, there is no particular limitation on a ratio between the two or more, which may be mixed in any ratio. Selecting the above types of salt containing the anion different from that of the 1-(hydroxyalkyl)-1′-alkyl viologen salt (1) is more conducive to combining the anions with the quaternary ammonium cations of viologen through ion exchange reaction, thereby forming the 1-(hydroxyalkyl)-1′-alkyl viologen salt (2).

In some embodiments of the present disclosure, the fourth solvent is at least one selected from the group consisting of deionized water, ethanol, NMP, DMF, and DMSO. The above solvent shows desirable solubility for 1-(hydroxyalkyl)-1′-alkyl viologen salt (1) and the salt containing the anion different from that of the 1-(hydroxyalkyl)-1′-alkyl viologen salt (1), thus promoting the ion exchange reaction to proceed fully.

In some embodiments of the present disclosure, a mass ratio of the 1-(hydroxyalkyl)-1′-alkyl viologen salt (1), the salt containing the anion different from that of the 1-(hydroxyalkyl)-1′-alkyl viologen salt (1), and the fourth solvent is in a range of (540-700):(470-1000):5000, preferably (550-570):(500-900):5000. Controlling the mass ratio of the 1-(hydroxyalkyl)-1′-alkyl viologen salt (1), the salt containing the anion different from that of the 1-(hydroxyalkyl)-1′-alkyl viologen salt (1), and the fourth solvent within the above range is more conducive to undergoing the ion exchange reaction between the salt containing the anion different from that of the 1-(hydroxyalkyl)-1′-alkyl viologen salt (1) with the 1-(hydroxyalkyl)-1′-alkyl viologen salt (1).

In the present disclosure, there is no particular limitation on an operation of mixing the 1-(hydroxyalkyl)-1′-alkyl viologen salt (1), the salt containing the anion different from that of the 1-(hydroxyalkyl)-1′-alkyl viologen salt (1), and the fourth solvent, and a technical scheme for preparing a mixed material well known to those skilled in the art may be used.

In some embodiments of the present disclosure, the ion exchange reaction is conducted at a temperature of 10° C. to 35° C., and preferably 20° C. to 30° C. In some embodiments, the ion exchange reaction is conducted for 1 h to 12 h, and preferably 8 h to 11 h.

In some embodiments of the present disclosure, a product obtained from the ion exchange reaction is mixed with a third precipitant, and a resulting mixture is subjected to filtration, followed by drying, to obtain the 1-(hydroxyalkyl)-1′-alkyl viologen salt (2).

In some embodiments of the present disclosure, the third precipitant is at least one of DCM, DCE, ethanol, diethyl ether, deionized water, and methanol. In some embodiments, when the third precipitant is a mixture of two or more of the above, there is no special limitation on a ratio between the two or more, which may be mixed in any ratio. In the present disclosure, by using the third precipitant, the 1-(hydroxyalkyl)-1′-alkyl viologen salt (2) could be separated from a reaction solution.

In some embodiments of the present disclosure, a mass ratio of the third precipitant to the product obtained by the ion exchange reaction is in a range of (4-11):1, and preferably (5-10):1.

In some embodiments of the present disclosure, the drying is conducted at a temperature of 50° C. to 70° C., and preferably 55° C. to 60° C. In some embodiments of the present disclosure, the drying is conducted for 20 h to 25 h, and preferably 24 h to 25 h.

In some embodiments of the present disclosure, after obtaining the 1-(hydroxyalkyl)-1′-alkyl viologen salt (2), the 1-(hydroxyalkyl)-1′-alkyl viologen salt (2) is mixed with a vinyl carbonyl compound, a catalyst, a polymerization inhibitor, and a fifth solvent, and a resulting mixture is subjected to esterification reaction, to obtain the viologen-based acrylate.

In some embodiments of the present disclosure, the vinyl carbonyl compound is at least one selected from the group consisting of 2-isocyanatoethyl acrylate, isocyanatoethyl methacrylate, acrylic acid, methacrylic acid, acryloyl chloride, and methacryloyl chloride. In the present disclosure, by selecting the vinyl carbonyl compound as described above, the viologen-based acrylate is formed.

In some embodiments, the catalyst is at least one selected from the group consisting of DBTDL, TEA, sodium carbonate, and potassium carbonate.

In some embodiments of the present disclosure, the polymerization inhibitor is at least one selected from the group consisting of p-methoxyphenol, p-benzoquinone, and p-hydroquinone. In some embodiments, when the polymerization inhibitor is a mixture of two or more of the above, there is no special limitation on a ratio between the two or more, which may be mixed in any ratio.

In some embodiments of the present disclosure, the fifth solvent is at least one selected from the group consisting of NMP, DMF, THF, 1,4-dioxane, acetonitrile, and chloroform. In some embodiment, when the fifth solvent is a mixture of two or more of the above, there is no special limitation on a ratio between the two or more, which may be mixed in any ratio.

In some embodiments of the present disclosure, a mass ratio of the 1-(hydroxyalkyl)-1′-alkyl viologen salt (2), the vinyl carbonyl compound, the catalyst, the polymerization inhibitor, and the fifth solvent is in a range of (600-1500):(130-310):(0.5-7.0):(1-2.5):5000, and preferably (610-1400):(140-300):(0.5-5.0):(1-1.5):5000.

In the present disclosure, there is no particular limitation on an operation of mixing the 1-(hydroxyalkyl)-1′-alkyl viologen salt (2), the vinyl carbonyl compound, the catalyst, the polymerization inhibitor, and the fifth solvent, and a technical scheme for preparing a mixed material well known to those skilled in the art may be adopted to make each component fully dissolved.

In some embodiments of the present disclosure, the esterification reaction is conducted at a temperature of 10° C. to 35° C., and preferably 20° C. to 30° C. In some embodiments, the esterification reaction is conducted for 1 h to 36 h, and preferably 20 h to 25 h. Controlling the temperature and time for the esterification reaction within the above range could avoid the occurrence of side reactions and ensure that the raw materials are fully reacted to form the viologen-based acrylate.

In some embodiments of the present disclosure, a product obtained from the esterification reaction is mixed with a fourth precipitant, and a resulting mixture is subjected to filtration, followed by drying, to obtain the viologen-based acrylate.

In some embodiments of the present disclosure, the fourth precipitant is at least one of DCE, ethanol, diethyl ether, and methanol.

In some embodiments of the present disclosure, a mass ratio of the fourth precipitant to the product obtained from the esterification reaction is in a range of (4-11):1, and preferably (5-10):1.

In some embodiments of the present disclosure, the drying is conducted at a temperature of 20° C. to 30° C., and preferably 25° C. to 30° C. In some embodiments, the drying is conducted for 15 h to 25 h, and preferably 18 h to 24 h.

In some embodiments of the present disclosure, the acrylate is at least one of ethyl methacrylate, ethyl acrylate, butyl acrylate, and butyl methacrylate. Selecting the above types of acrylate could not only improve the flexibility of the binder and soften the electrode, but also enhance the hydrophobicity of the binder, thereby avoiding the decrease in battery capacity and recycling performance caused by the absorbed water.

In the present disclosure, on the one hand, the chain segment of poly(ethylene glycol) methyl ether acrylate could enhance the flexibility of the binder; on the other hand, due to desirable lithium ion transportation, this chain segment could improve the ion transportation between the electrode and the electrolyte interface and reduce the interface impedance.

In some embodiments of the present disclosure, the initiator is at least one of AIBN, ACCN, dimethyl 2,2′-azobisisobutyrate, and BPO. In some embodiments, when the initiator is a mixture of two or more of the above, there is no special limitation on a ratio between the two or more, which may be mixed in any ratio.

In some embodiments of the present disclosure, a mass of the initiator accounts for 0.05% to 0.15% of a total mass of the viologen-based acrylate, and at least one of the acrylate and poly(ethylene glycol) methyl ether acrylate. In the present disclosure, controlling the amount of the initiator within the above range could improve the free radical polymerization.

In some embodiments of the present disclosure, the first solvent is at least one selected from the group consisting of acetonitrile, toluene, acetone, NMP, DMF, THF, 1,4-dioxane, and chloroform. In some embodiments, when the first solvent is a mixture of two or more of the above, there is no special limitation on a ratio between the two or more, which may be mixed in any ratio. There is no particular limitation on the amount of the first solvent, as long as a total mass concentration of the viologen-based acrylate, at least one of the acrylate and poly(ethylene glycol) methyl ether acrylate is within the range of 20% to 40%.

In some embodiments of the present disclosure, a mass ratio of the viologen-based acrylate, the acrylate, poly(ethylene glycol) methyl ether acrylate, the initiator, and the first solvent during the free radical polymerization is in a range of (100-500):(0-600):(0-800):(0.3-1.5):(1000-5000), and preferably (200-500):(0-580):(0-700):(0.4-1.3):(1000-2800). Controlling each component within the above range is more conducive to forming an ionic polymer binder with excellent bonding properties.

In some embodiments of the present disclosure, the free radical polymerization is conducted at a temperature of 50° C. to 80° C., and preferably 65° C. to 75° C. In some embodiments, the free radical polymerization is conducted for 8 h to 12 h, and preferably 9 h to 11 h. Controlling the above parameters of the free radical polymerization could further improve polymerization.

In some embodiments of the present disclosure, the free radical polymerization is conducted in inert atmosphere. In some embodiments, a gas for providing the inert atmosphere is nitrogen and/or argon. The inert atmosphere could avoid the interference of air on the free radical polymerization to ensure that a polymer obtained by the free radical polymerization has good properties.

In some embodiments of the present disclosure, a product obtained from the free radical polymerization is mixed with a fifth precipitant, and a resulting mixture is subjected to filtration, followed by drying, to obtain the viologen-based ionic polymer binder.

In some embodiments of the present disclosure, the fifth precipitant is at least one of diethyl ether, ethanol, acetone, and methanol. In some embodiments, when the fifth precipitant is a mixture of two or more of the above, there is no special limitation on a ratio between the two or more, which may be mixed in any ratio. In some embodiments, a volume ratio of the fifth precipitant to the system obtained from the free radical polymerization is in a range of (4-11):1, and preferably (5-10):1.

In some embodiments of the present disclosure, the drying is conducted at a temperature of 30° C. to 60° C., and preferably 50° C. to 60° C. In some embodiments, the drying is conducted for 1 h to 24 h, and preferably 12 h to 24 h.

In the present disclosure, the preparation method is simple to operate and can introduce viologen-based acrylate into the ionic polymer binder. An electrostatic interaction between the active material and the quaternary ammonium cations and the counter anions in the viologen structure could enhance the adhesion between active materials; the counter anions could promote the transportation of lithium ions; the viologen has a stable structure and reversible redox properties. When the ionic polymer is used as a binder for the cathode of a lithium-ion battery, it is beneficial to improve the capacity retention and recycling stability of the battery. The acrylate could not only improve the flexibility of the binder and soften the electrode, but also enhance the hydrophobicity of the binder, thereby avoiding the decrease in battery capacity and recycling performance caused by the absorbed water. On the one hand, the poly(ethylene glycol) methyl ether acrylate could enhance the flexibility of the binder; on the other hand, due to desirable lithium ion transportation, this component could improve the ion transportation between the electrode and the electrolyte interface, and thereby reduce the interface impedance.

The present disclosure further provides use of the viologen-based ionic polymer binder as described in the above technical solutions or the viologen-based ionic polymer binder prepared by the method as described in the above technical solutions in the preparation of an electrode of a lithium-ion battery.

In some embodiments of the present disclosure, the electrode of the lithium-ion battery is one selected from the group consisting of a cathode and an anode.

In some embodiments of the present disclosure, the electrode of the lithium-ion battery is prepared by a process including the following steps:
1) dissolving the viologen-based ionic polymer binder in an organic solvent to obtain a binder solution;
2) mixing an electrode active material and a conductive material to obtain a mixed powder;
3) mixing the binder solution obtained in step 1) and the mixed powder obtained in step 2) with an organic solvent to obtain an electrode slurry; and
4) applying the electrode slurry obtained in step 3) onto a current collector to obtain the electrode of the lithium-ion battery.

In some embodiments of the present disclosure, the viologen-based ionic polymer binder is dissolved in an organic solvent to obtain a binder solution.

In some embodiments of the present disclosure, the organic solvent is one or more of NMP, DMF, DMSO, and acetonitrile.

In the present disclosure, there is no particular limitation on the amount of the organic solvent, as long as the binder solution has a viscosity of 0.1-20 Pa s, preferably 10-15 Pa s.

In some embodiments of the present disclosure, the dissolution of the viologen-based ionic polymer binder in the organic solvent is performed at 25° C. In some embodiments, dissolving the viologen-based ionic polymer binder in the organic solvent is performed for 8 h to 12 h, and preferably 10 h to 12 h.

In some embodiments of the present disclosure, an active material is mixed with a conductive material to obtain a mixed powder.

In some embodiments of the present disclosure, the active material is a cathode active material. In some embodiments, the cathode active material includes one of lithium iron phosphate, lithium cobalt oxide, lithium manganate, ternary nickel cobalt manganese 811, ternary nickel cobalt manganese 523, ternary nickel cobalt aluminum 811, ternary nickel cobalt manganese 622, ternary nickel cobalt manganese 613, and lithium titanate. The use of the cathode active material of the above types could enable the lithium-ion battery to have desirable electrochemical performance.

In some embodiments of the present disclosure, the conductive material includes one or more of superconducting carbon black, carbon nanotubes, acetylene black, and Ketjen black. The above-mentioned types of conductive materials could make lithium-ion battery have desirable electrochemical properties.

In some embodiments of the present disclosure, mixing the cathode active material and the conductive material includes: at room temperature, ball milling the cathode active material and the conductive material at a rotation speed of 600 rpm to 1,200 rpm for 0.5 h to 4 h, preferably at a rotation speed of 900 rpm to 1,100 rpm for 0.5 h to 1.5 h. The above mixing method could make the cathode active material and the conductive material mixed uniformly.

In some embodiments of the present disclosure, after obtaining the binder solution and the mixed powder, the binder solution and the mixed powder are mixed with an organic solvent to obtain an electrode slurry.

In some embodiments of the present disclosure, the organic solvent is one or more of NMP, DMF, DMSO, and acetonitrile. The above organic solvent allows the electrode slurry to be mixed more uniformly.

In some embodiments of the present disclosure, a mass ratio of the cathode active material, the conductive material, and the viologen-based ionic polymer binder in the electrode slurry is in a range of (60-100):(1-20):(1-20), more preferably (75-96):(2-15):(2-15).

There is no particular limitation on the amount of the organic solvent, which could be adjusted as needed. In some embodiments, when a mass ratio of the cathode active material, the conductive material, and the viologen-based ionic polymer binder in the electrode slurry is in a range of (60-100):(1-20):(1-20), a total mass concentration of the mixed powder and the binder in the electrode slurry is in a range of 30 wt % to 95 wt %, and preferably 30 wt % to 85 wt %. Controlling the concentration of the electrode slurry within the above range could make the electrode slurry easier to be applied and make the obtained cathode have more uniform thickness.

In the present disclosure, there is no particular limitation on means for mixing the binder solution, the mixed powder, and the organic solvent, and any conventional means for mixing may be used to form a uniform electrode slurry. In some embodiments, mixing the binder solution, the mixed powder, and the organic solvent includes: ball milling the mixed powder and the binder solution in a ball mill at a rotation speed of 900 rpm to 1,100 rpm for 2 h to 4 h, preferably 1,000 rpm to 1,100 rpm for 2 h to 3 h; and adding the organic solvent thereto and continuing the ball milling for 1 h to 3 h, preferably 2 h to 3 h. The above mixing operation and the parameters within the above range are more conducive to uniform mixing of the raw materials.

In some embodiments of the present disclosure, after obtaining the electrode slurry, the electrode slurry is applied onto a current collector to obtain the electrode of the lithium-ion battery.

In some embodiments of the present disclosure, the current collector is an aluminum foil. There is no special limitation on a size of the aluminum foil, which could be adjusted according to actual demands.

In the present disclosure, there is no particular limitation on the applying method, and any conventional applying method may be used. In some embodiments, the applying is performed by scrape coating. There is no particular limitation on a type of the scraper used for the scrape coating, and any instrument or equipment well known to those skilled in the art may be used.

In the present disclosure, there is no particular limitation on the amount of the electrode slurry applied onto the current collector, which could be adjusted as needed. In some embodiments, the electrode slurry is in an amount such that a thickness of a slurry layer obtained on the cathode ranges from 60 m to 250 m.

In some embodiments of the present disclosure, a product obtained by the applying is subjected to drying, rolling, and cutting into pieces in sequence to obtain the electrode of the lithium-ion battery. Through the drying, rolling and cutting, a flat cathode with a suitable size could be obtained.

In some embodiments of the present disclosure, the drying includes atmospheric pressure drying or vacuum drying. In some embodiments, the atmospheric pressure drying is conducted at a temperature of 50° C. to 90° C., and preferably 60° C. to 80° C. In some embodiments, the atmospheric pressure drying is conducted for 6 h to 10 h, and preferably 8 h to 9 h. In some embodiments, the vacuum drying is conducted at a temperature of 70° C. to 90° C., and preferably 80° C. to 90° C. In some embodiments, the vacuum drying is conducted for 8 h to 15 h, and preferably 9 h to 12 h. Controlling the drying temperature and time within the above range is conducive to the full volatilization of the organic solvent and could avoid cracking and curling defects of the cathode of the lithium-ion battery caused by the volatilization of the organic solvent.

In the present disclosure, there is no special limitation on the operations of rolling and cutting, and operations well known to those skilled in the art may be adopted.

In the present disclosure, the viologen-based ionic polymer binder has a viologen-based polyacrylate chain segment, a polyacrylate chain segment, and a poly(ethylene glycol) methyl ether acrylate chain segment. It results in the lithium-ion battery assembled with the cathode prepared by using the viologen-based ionic polymer binder shows low interface impedance, high specific capacity, high capacity retention, and desirable recycling stability.

The technical solutions of the present disclosure will be clearly and completely described below in conjunction with examples of the present disclosure. Apparently, the described examples are merely a part rather than all of the examples of the present disclosure. All other examples obtained by those of ordinary skill in the art based on the examples of the present disclosure without creative efforts shall fall within the scope of the present disclosure.

### Example 1

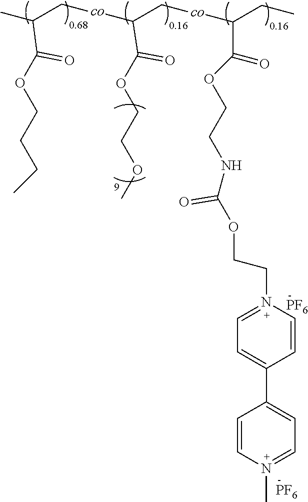

Provided was a viologen-based ionic polymer binder having a chemical structure shown in formula I. In formula I, R₁, R₃, and R₄ were hydrogen; R₂ was butyl; R₅ was methyl; X was ethyl dimethylidenecarbamate group; Y was PF₆; m=0.68; n=0.16; p=9; and q=0.16.

The viologen-based ionic polymer binder was prepared according to the following procedures:

In nitrogen atmosphere, 500 g of viologen phosphate acrylate, 420 g of butyl acrylate, 373 g of poly(ethylene glycol) methyl ether acrylate, 1.29 g of AIBN, and 3,000 g of acetonitrile were mixed in a reactor, and a resulting mixture was subjected to free radical polymerization at 70° C. for 10 h. A resulting ionic polymer solution was poured into 45,000 g of ethanol, and the ionic polymer was precipitated. A resulting system was subjected to filtration, and a resulting residual product was dried in a vacuum drying oven at 50° C. for 12 h to obtain 1,150 g of the viologen-based ionic polymer binder, named A1.

The viologen phosphate acrylate referred to 1-(2-(((2-(acryloyloxy)ethyl)carbamoyl)oxy)ethyl)-1′-methylviologen hexafluorophosphate having a structural formula as follows:

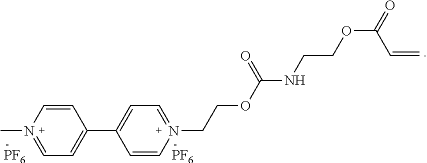

The viologen phosphate acrylate was prepared according to the following procedures:
(1) 200 g of 4,4′-bipyridine and 218 g of methyl iodide were dissolved in 4,000 g of DCM, and a resulting mixture was stirred at 25° C. for 12 h, i.e., undergoing a first alkylation reaction. After the reaction, the resulting mixture was poured into 44,180 g of n-hexane for precipitation. A resulting mixture was subjected to filtration, and a resulting residual product was vacuum-dried at 50° C. for 12 h to obtain 395 g of yellow solid, which was N-methyl-4,4′-bipyridine iodide.
(2) The N-methyl-4,4′-bipyridine iodide obtained in step (1) and 199 g of 2-bromoethanol were mixed with 4,000 g of acetonitrile and dissolved therein, and a resulting mixture was subjected to a second alkylation reaction at 80° C. for 60 h. A resulting mixture was poured into 51,000 g of DCM for precipitation. A resulting mixture was subjected to filtration, and a resulting residual product was vacuum-dried at 50° C. for 12 h to obtain 552 g of yellow solid, which was 1-(hydroxyalkyl)-1′-alkyl viologen salt (1), namely 1-(2-hydroxyethyl)-1′-methylviologen salt.
(3) The 1-(hydroxyalkyl)-1′-alkyl viologen salt (1) obtained in step (2) and 510 g of ammonium hexafluorophosphate were mixed with 5,000 g of deionized water and dissolved therein. A resulting mixture was stirred at 25° C. for 10 h, i.e., undergoing ion exchange reaction. After the reaction, a resulting mixture was poured into 60,620 g of deionized water for precipitation. A resulting mixture was subjected to filtration, and a resulting residual product was vacuum-dried at 60° C. for 24 h to obtain 620 g of white solid, which was 1-(hydroxyalkyl)-1′-alkyl viologen salt (2), namely 1-(2-hydroxyethyl)-1′-methylviologen hexafluorophosphate.
(4) In a nitrogen atmosphere, the 1-(hydroxyalkyl)-1′-alkyl viologen salt (2) obtained in step (3), 208 g of 2-isocyanatoethyl acrylate, 0.77 g of DBTDL, and 1.32 g of p-benzoquinone were mixed with 5,000 g of 1,4-dioxane and dissolved therein, and a resulting mixture was stirred at 25° C. for 24 h, undergoing esterification reaction. After the reaction, a resulting mixture was poured into 58,300 g of ethanol for precipitation. A resulting mixture was subjected to filtration, and a resulting residual product was vacuum-dried at 25° C. for 24 h to obtain 719 g of white solid, which was the viologen phosphate acrylate, namely 1-(2-(((2-(acryloyloxy)ethyl)carbamoyl)oxy)ethyl)-1′-methylviologen hexafluorophosphate.

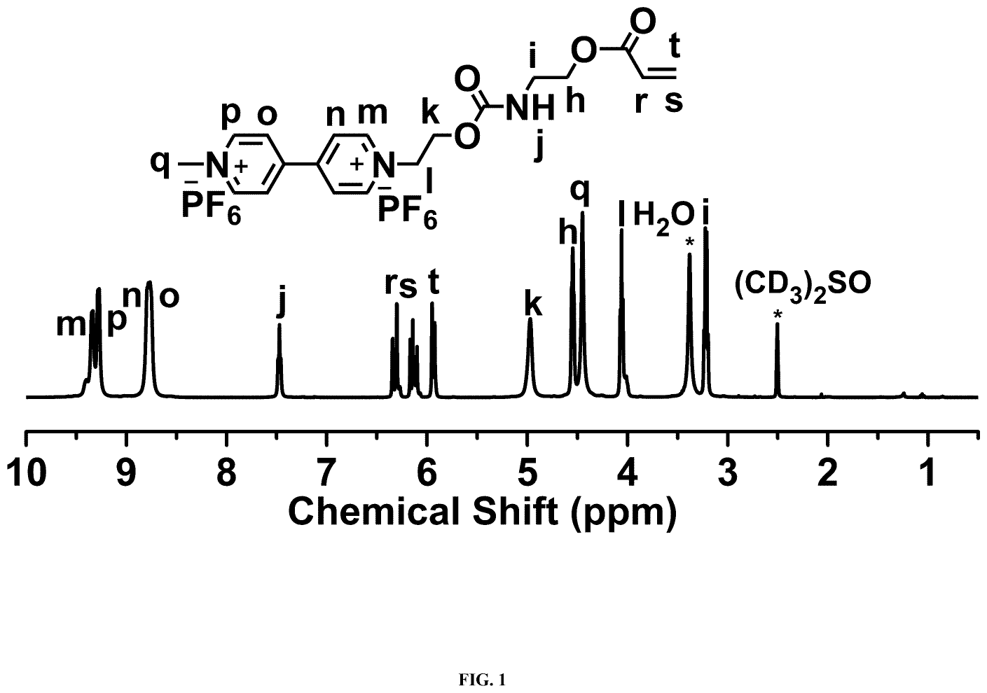

FIG. 1 shows an ¹H-NMR spectrum of the 1-(2-(((2-(acryloyloxy)ethyl)carbamoyl)oxy)ethyl)-1′-methylviologen hexafluorophosphate prepared in this example.

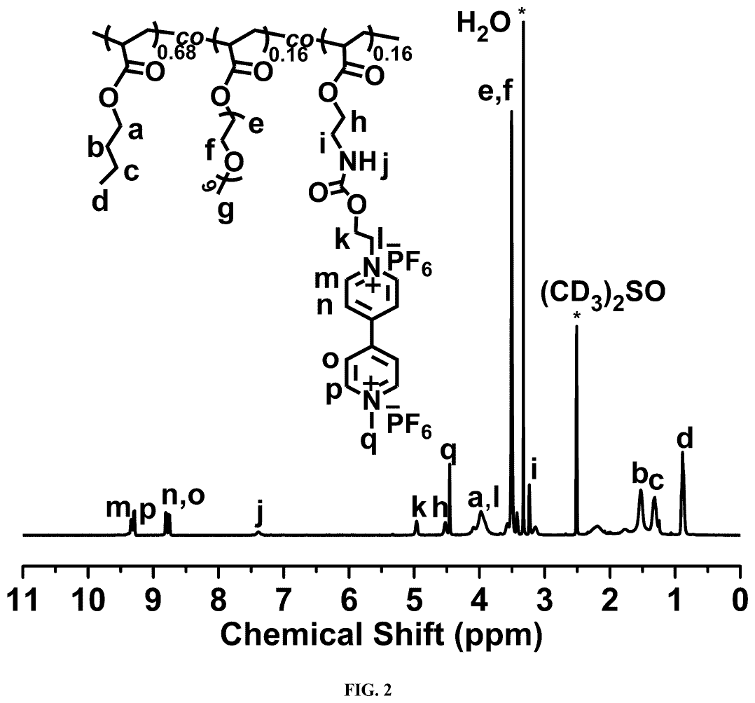

FIG. 2 shows an ¹H-NMR spectrum of the viologen-based ionic polymer binder A1 prepared in this example.

### Example 2

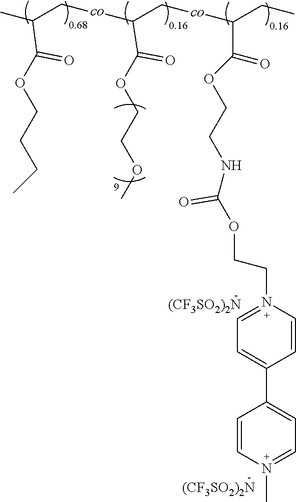

Provided was a viologen-based ionic polymer binder having a chemical structure shown in formula I. In formula I, R₁, R₃, and R₄ were hydrogen; R₂ was butyl; R₅ was methyl; X was ethyl dimethylidenecarbamate group; Y was (CF₃SO₂)₂N; m=0.68; n=0.16; p=9; and q=0.16.

The viologen-based ionic polymer binder was prepared according to the following procedures:

In a nitrogen atmosphere, 500 g of viologen bis(trifluoromethanesulfonyl)imide acrylate, 277 g of butyl acrylate, 246 g of poly(ethylene glycol) methyl ether acrylate, 1.02 g of AIBN, and 2,377 g of acetonitrile were mixed in a reactor, and a resulting mixture was subjected to free radical polymerization at 70° C. for 10 h. After the reaction, a resulting ionic polymer solution was poured into 10,230 g of ethanol for precipitation. A resulting mixture was subjected to filtration and a resulting residual product was dried in a vacuum drying oven at 50° C. for 12 h to obtain 950 g of the viologen-based ionic polymer binder, named A2.

The viologen bis(trifluoromethanesulfonyl)imide acrylate was 1-(2-(((2-(acryloyloxy)ethyl)carbamoyl)oxy)ethyl)-1′-methylviologen bis(trifluoromethanesulfonyl)imide having a structural formula as follows:

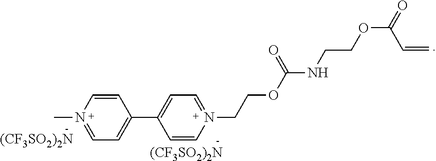

The viologen bis(trifluoromethanesulfonyl)imide acrylate was prepared according to the procedures:
(1) 552 g of 1-(hydroxyalkyl)-1′-alkyl viologen salt (1) and 898 g of lithium bis(trifluoromethanesulfonyl)imide were mixed with 5,000 g of deionized water and dissolved therein, and a resulting mixture was stirred at 25° C. for 10 h, i.e., undergoing ion exchange reaction. A resulting mixture was poured into 82,800 g of deionized water for precipitation. A resulting mixture was subjected to filtration and a resulting residual product was vacuum-dried at 60° C. for 24 h to obtain 1,370 g of white solid, which was 1-(hydroxyalkyl)-1′-alkyl viologen salt (2), namely 1-(2-hydroxyethyl)-1′-methylviologen bis(trifluoromethanesulfonyl)imide.
(2) In a nitrogen atmosphere, the 1-(hydroxyalkyl)-1′-alkyl viologen salt (2) obtained in step (1), 293 g of 2-isocyanatoethyl acrylate, 0.85 g of DBTDL, and 1.43 g of p-benzoquinone were mixed with 5,000 g of 1,4-dioxane and dissolved therein, and a resulting mixture was stirred at 25° C. for 24 h, i.e., undergoing esterification reaction. After the reaction, a resulting mixture was poured into 16,700 g of ethanol for precipitation. A resulting mixture was subjected to filtration and a resulting residual product was vacuum-dried at 25° C. for 24 h to obtain 1,532 g of white solid, which was the viologen bis(trifluoromethanesulfonyl)imide acrylate, namely 1-(2-(((2-(acryloyloxy)ethyl)carbamoyl)oxy)ethyl)-1′-methylviologen bis(trifluoromethanesulfonyl) imide.

### Example 3

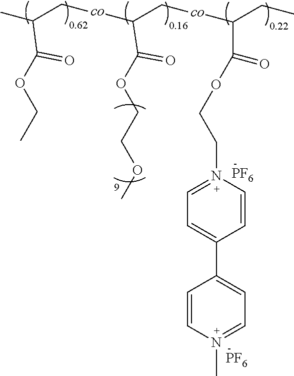

Provided was a viologen-based ionic polymer binder having a chemical structure shown in formula I. In formula I, R₁, R₃, and R₄ were hydrogen; R₂ was ethyl; R₅ was methyl; X was dimethylene group; Y was PF₆; m=0.62; n=0.16; p=9; and q=0.22.

The viologen-based ionic polymer binder was prepared according to the following procedures:

In an argon atmosphere, 450 g of viologen phosphate acrylate, 94 g of ethyl acrylate, 277 g of poly(ethylene glycol) methyl ether acrylate, 0.82 g of AIBN, and 1,860 g of NMP were mixed in a reactor and a resulting mixture was subjected to free radical polymerization at 70° C. for 10 h. After the polymerization, a resulting ionic polymer solution was poured into 32,000 g of ethanol for precipitation. A resulting mixture was subjected to filtration, and a resulting residual product was dried in a vacuum drying oven at 50° C. for 12 h to obtain 805 g of the viologen-based ionic polymer binder, named A3.

The viologen phosphate acrylate was 1-((2-acryloyloxy)ethyl)-1′-methylviologen hexafluorophosphate having a structural formula as follows:

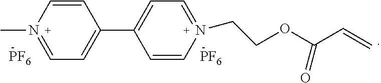

The viologen phosphate acrylate was prepared according to the following procedures:
(1) 230 g of 4,4′-bipyridine and 223 g of DMS were dissolved in 4,000 g of DCM, and a resulting mixture was stirred at 25° C. for 12 h, i.e., undergoing a first alkylation reaction. A resulting mixture was poured into 44,530 g of n-hexane for precipitation. A resulting system was subjected to filtration and a resulting residual product was vacuum-dried at 50° C. for 12 h to obtain 405 g of light yellow solid, which was N-methyl-4,4′-bipyridine sulfate.
(2) The N-alkyl-4,4′-bipyridine salt obtained in step (1) and 227 g of 2-bromoethanol were mixed with 4,000 g of NMP and dissolved therein, and a resulting mixture was subjected to a second alkylation reaction at 90° C. for 60 h. A resulting mixture was poured into 51,000 g of DCM for precipitation. A resulting system was subjected to filtration and a resulting residual product was vacuum-dried at 50° C. for 12 h to obtain 563 g of light yellow solid, which was 1-(hydroxyalkyl)-1′-alkyl viologen salt (1), namely 1-(2-hydroxyethyl)-1′-methylviologen salt.
(3) The 1-(hydroxyalkyl)-1′-alkyl viologen salt (1) obtained in step (2) and 557 g of ammonium hexafluorophosphate were mixed with 5,000 g of deionized water and dissolved therein, and a resulting mixture was stirred at 25° C. for 10 h, i.e., undergoing ion exchange reaction. After the reaction, a resulting mixed system was poured into 60,000 g of deionized water for precipitation. A resulting mixture was subjected to filtration, and a resulting residual product was vacuum-dried at 60° C. for 24 h to obtain 690 g of white solid, which was 1-(hydroxyalkyl)-1′-alkyl viologen salt (2), namely 1-(2-hydroxyethyl)-1′-methylviologen hexafluorophosphate.
(4) In a nitrogen atmosphere, the 1-(hydroxyalkyl)-1′-alkyl viologen salt (2) obtained in step (3), 149 g of acryloyl chloride, 4.9 g of TEA, and 1.49 g of p-benzoquinone were mixed with 5,000 g of 1,4-dioxane, and a resulting mixture was stirred at 25° C. for 24 h, i.e., undergoing esterification reaction. After the reaction, a resulting mixture was poured into 58,500 g of ethanol for precipitation. A resulting mixture was subjected to filtration, and a resulting residual product was vacuum-dried at 25° C. for 24 h to obtain 839 g of white solid, which was the viologen phosphate acrylate, namely 1-((2-acryloyloxy)ethyl)-1′-methylviologen hexafluorophosphate.

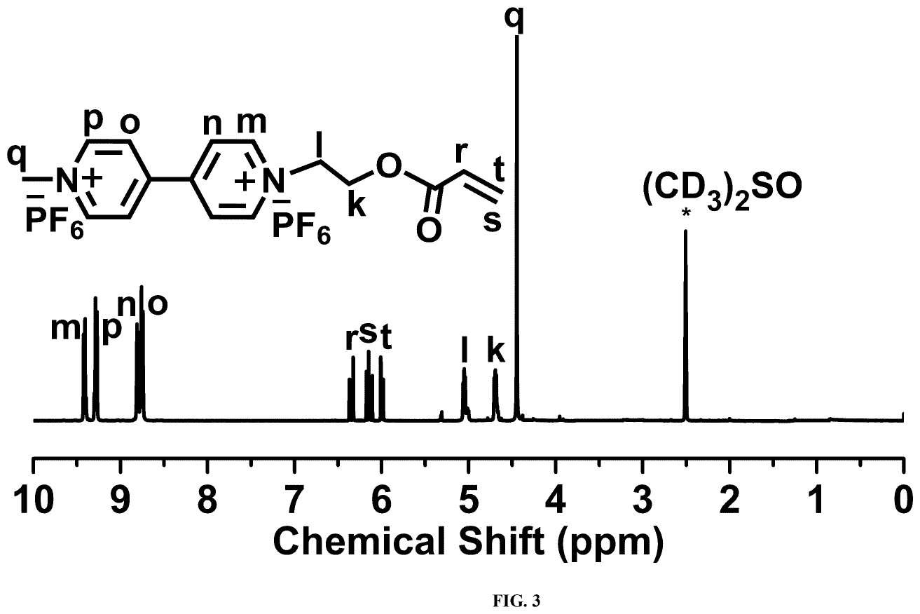

FIG. 3 shows an H-NMR spectrum of 1-((2-acryloyloxy)ethyl)-1′-methylviologen hexafluorophosphate prepared in this example.

### Example 4

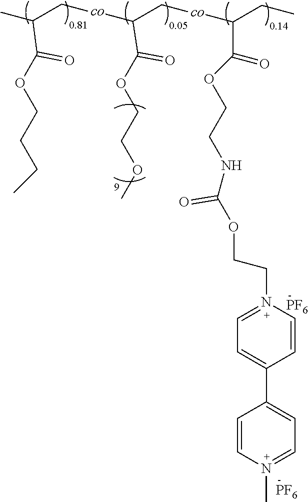

Provided was a viologen-based ionic polymer binder having a chemical structure shown in formula I. In formula I, R₁, R₃, and R₄ were hydrogen; R₂ was butyl; R₅ was methyl; X was ethyl dimethylidenecarbamate group; Y was PF₆; m=0.81; n=0.05; p=9; and q=0.14.

The viologen-based ionic polymer binder was prepared according to the procedures:

In a nitrogen atmosphere, 500 g of viologen phosphate acrylate (this reactant was the same as that in Example 1), 573 g of butyl acrylate, 134 g of poly(ethylene glycol) methyl ether acrylate, 1.21 g of ACCN, and 2,800 g of acetonitrile were mixed in a reactor, and a resulting mixture was subjected to free radical polymerization at 70° C. for 10 h. A resulting ionic polymer solution was poured into 42,000 g of ethanol for precipitation. A resulting mixture was subjected to filtration, and a resulting residual product was dried in a vacuum drying oven at 50° C. for 12 h to obtain 1,050 g of the viologen-based ionic polymer binder, named A4.

### Example 5

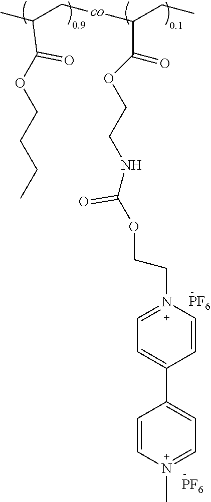

Provided was a viologen-based ionic polymer binder having a chemical structure shown in formula I. In formula I, R₁ and R₄ were hydrogen; R₂ was butyl; R₅ was methyl; X was ethyl dimethylidenecarbamate group; Y was PF₆; m=0.9; n=0; p=0; and q=0.1.

The viologen-based ionic polymer binder was prepared according to the procedures:

In nitrogen atmosphere, 200 g of viologen phosphate acrylate (this reactant was the same as that in Example 1), 356 g of butyl acrylate, 0.56 g of AIBN, and 1,280 g of acetonitrile were mixed in a reactor, and a resulting mixture was subjected to free radical polymerization at 70° C. for 10 h. A resulting ionic polymer solution was poured into 16,000 g of ethanol for precipitation. A resulting mixture was subjected to filtration, and a resulting residual product was dried in a vacuum drying oven at 50° C. for 12 h to obtain 530 g of the viologen-based ionic polymer binder, named A5.

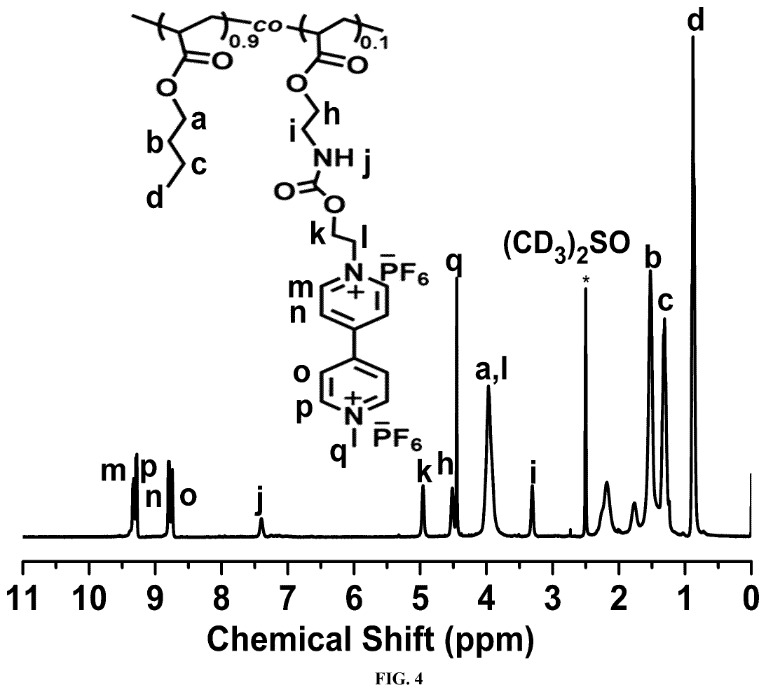

FIG. 4 shows an ¹H-NMR spectrum of the viologen-based ionic polymer binder A5 prepared in this example.

### Example 6

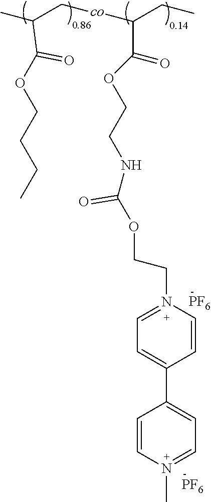

Provided was a viologen-based ionic polymer binder having a chemical structure shown in formula I. In formula I, R₁ and R₄ were hydrogen; R₂ was butyl; R₅ was methyl; X was ethyl dimethylidenecarbamate group; Y was PF₆; m=0.86; n=0; p=0; and q=0.14.

The viologen-based ionic polymer binder was prepared according to the procedures:

In nitrogen atmosphere, 200 g of viologen phosphate acrylate (this reactant was the same as that in Example 1), 243 g of butyl acrylate, 0.44 g of AIBN, and 1,008 g of acetonitrile were mixed in a reactor, and a resulting mixture was subjected to free radical polymerization at 70° C. for 10 h. A resulting ionic polymer solution was poured into 15,000 g of ethanol for precipitation. A resulting mixture was subjected to filtration, and a resulting residual product was dried in a vacuum drying oven at 50° C. for 12 h to obtain 429 g of the viologen-based ionic polymer binder, named A6.

### Example 7

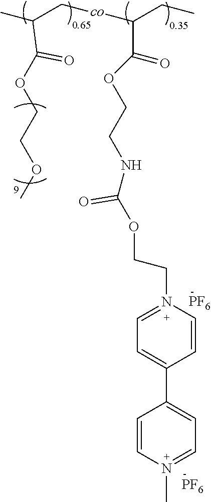

Provided was a viologen-based ionic polymer binder having a chemical structure shown in formula I. In formula I, R₃ and R₄ were hydrogen; R₅ was methyl; X was ethyl dimethylidenecarbamate group; Y was PF₆; m=0; n=0.65; p=9; and q=0.35.

The viologen-based ionic polymer binder was prepared according to the procedures:

In argon atmosphere, 500 g of viologen phosphate acrylate (this reactant was the same as that in Example 1), 696 g of poly(ethylene glycol) methyl ether acrylate, 1.19 g of AIBN, and 2,790 g of NMP were mixed in a reactor, and a resulting mixture was subjected to free radical polymerization at 70° C. for 10 h. A resulting ionic polymer solution was poured into 35,000 g of ethanol for precipitation. A resulting mixture was subjected to filtration, and a resulting residual product was dried in a vacuum drying oven at 50° C. for 12 h to obtain 1,100 g of the viologen-based ionic polymer binder, named A7.

### Example 8

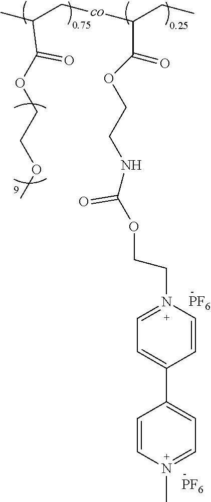

Provided was a viologen-based ionic polymer binder having a chemical structure shown in formula I. In formula I, R₃ and R₄ were hydrogen; R₅ was methyl; X was ethyl dimethylidenecarbamate group; Y was PF₆; m=0; n=0.75; p=9; and q=0.25.

The viologen-based ionic polymer binder was prepared according to the procedures:

In argon atmosphere, 250 g of viologen phosphate acrylate (this reactant was the same as that in Example 1), 562 g of poly(ethylene glycol) methyl ether acrylate, 0.81 g of AIBN, and 1,815 g of NMP were mixed in a reactor, and a resulting mixture was subjected to free radical polymerization at 70° C. for 10 h. After the reaction, a resulting ionic polymer solution was poured into 37,500 g of ethanol for precipitation. A resulting system was subjected to filtration and a resulting residual product was dried in a vacuum drying oven at 50° C. for 12 h to obtain 758 g of the viologen-based ionic polymer binder, named A8.

### Example 9

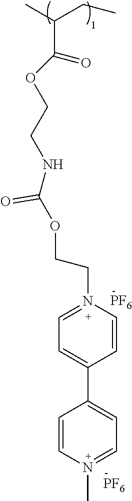

Provided was a viologen-based ionic polymer binder having a chemical structure shown in formula I. In formula I, R₄ was hydrogen; R₅ was methyl; X was ethyl dimethylidenecarbamate group; Y was PF₆; m=0; n=0; p=0; and q=1.

The viologen-based ionic polymer binder was prepared according to the procedures:

In argon atmosphere, 500 g of viologen phosphate acrylate (this reactant was the same as that in Example 1), 0.5 g of AIBN, and 1,166 g of DMF were mixed in a reactor, and a resulting mixture was subjected to free radical polymerization at 70° C. for 10 h. After the reaction, a resulting ionic polymer solution was poured into 17,000 g of ethanol for precipitation. A resulting mixture was subjected to filtration, and a resulting residual product was dried in a vacuum drying oven at 50° C. for 12 h to obtain 460 g of the viologen-based ionic polymer binder, named A9.

### Comparative Example 1

300 g of a dry PVDF (HSV900) powder and 2,700 g of NMP were mixed in a reactor, and a resulting mixture was stirred at 25° C. for 10 h to obtain a PVDF binder solution, recorded as B1.

### Comparative Example 2

400 g of a sodium carboxymethyl cellulose powder (MAC500LC, Shenzhen Kejing New Materials Co., Ltd, China), 1,712 g of styrene-butadiene rubber latex (S2919, Shenzhen Kejing New Materials Co., Ltd, China, with a polymer mass fraction of 35%), and 9,000 g of pure water (with resistivity greater than 0.1 MΩ·cm) were mixed in a reactor, and a resulting mixture was stirred at 25° C. for 10 h to obtain a binder solution, recorded as B2.

### Use Examples 1 to 9

Cathodes of a lithium-ion battery were prepared using viologen-based ionic polymer binders prepared in Examples 1 to 9, and the specific steps were as follows:
1) 10 g of the viologen-based ionic polymer binder and 90 g of NMP were stirred in a container at 25° C. for 10 h until the polymer was completely dissolved to obtain a binder solution.
2) 80 g of lithium iron phosphate and 10 g of superconducting carbon black were ball-milled at a rotation speed of 1,032 rpm for 1 h to obtain a mixed powder.
3) 100 g of the binder solution obtained in step 1) and the mixed powder obtained in step 2) were ball-milled at a rotation speed of 1,032 rpm for 3 h, 80 g of NMP was then added thereto, and the ball milling was continued for 2 h to obtain an electrode slurry.
4) The electrode slurry obtained in step 3) was applied onto aluminum foil using a scraper; the coated aluminum foil was dried at 60° C. and atmospheric pressure for 8 h, then dried at 80° C. and atmospheric pressure for 8 h, and finally dried at 80° C. under vacuum for 12 h. The resulting aluminum foil was then rolled and cut into pieces to obtain lithium iron phosphate cathodes C1 to C9 (the cathode prepared with the viologen-based ionic polymer binder A1 in Example 1 was denoted as C1, and the remaining were similarly recorded as C2-C9, respectively).

### Comparative Use Example 1

A cathode for a lithium-ion battery was prepared using PVDF binder B1 prepared in Comparative Example 1. This example differed from Use Example 1 in that: B1 prepared in Comparative Example 1 was used as a binder solution, and other steps were the same as those in Use Example 1. A lithium iron phosphate cathode was obtained, recorded as C10.

### Comparative Use Example 2

An artificial graphite anode for a lithium-ion battery was prepared using the binder B2 prepared in Comparative Example 2, and the specific steps were as follows: 188 g of artificial graphite and 6 g of superconducting carbon black were ball-milled at a rotation speed of 1,032 rpm for 1 h to obtain a mixed powder. 60 g of the binder solution B2 prepared in Comparative Example 2 and the mixed powder were ball-milled at a rotation speed of 1,032 rpm for 3 h, and 136 g of pure water (with resistivity greater than 0.1 MQ cm) was added thereto, and the ball milling was continued for 2 h to obtain an anode slurry. The slurry was applied and dried according to the method of step 4) in Use Example 1 to obtain an artificial graphite anode for a lithium-ion battery, denoted as C11.

### Test Example 1

The viologen-based ionic polymer binders prepared in Examples 1 to 9 and the binder prepared in Comparative Example 1 were subjected to the following performance tests:

A viscosity average molecular weight M_{η} was determined according to the test method of GB/T 10247-2008.

A thermal decomposition temperature was measured by a thermogravimetric analyzer (Netzsch, Germany, TG209) in a nitrogen atmosphere from 25° C. to 600° C. at a raising rate of 10° C.·min⁻¹.

A glass transition temperature was measured by differential scanning calorimeter (TA DSC Q100) at a temperature ranging from −80° C. to 180° C. in a nitrogen atmosphere with a heating/cooling rate of ±10° C.·min⁻¹. The performance test results of the binders were shown in Table 1.

TABLE 1
Performance indicators of binder

| Binder | M_{η} (Da) | T_{d} (° C.) | T_{g} (° C.) |
|---|---|---|---|
| A1 | 3.9 × 10⁴ | 318.7 | −43.5 |
| A2 | 4.0 × 10⁴ | 317.8 | −45.3 |
| A3 | 3.3 × 10⁴ | 323.5 | −44.6 |
| A4 | 2.8 × 10⁴ | 322.5 | −44.7 |
| A5 | 3.3 × 10⁴ | 326.3 | −45.1 |
| A6 | 3.2 × 10⁴ | 324.2 | −43.8 |
| A7 | 4.1 × 10⁴ | 326.3 | −44.4 |
| A8 | 3.8 × 10⁴ | 325.7 | −43.2 |
| A9 | 2.0 × 10⁴ | 325.5 | 24.5 |
| B1 | 5.0 × 10⁵ | 316.0 | −47.0 |

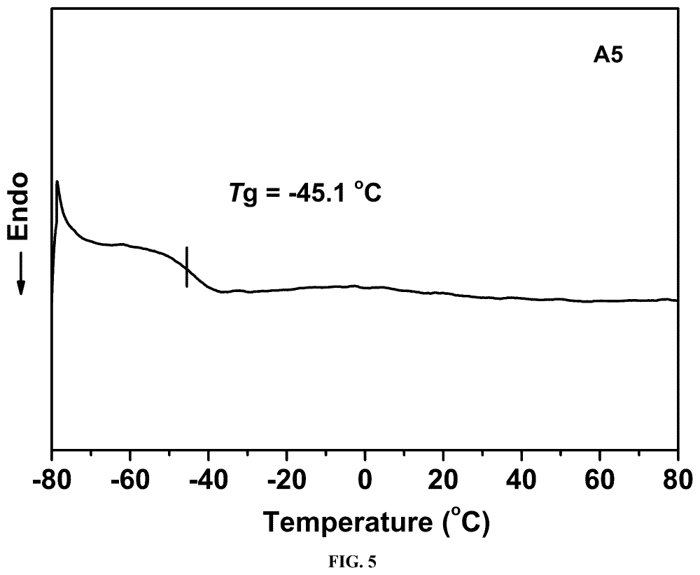

FIG. 5 shows a DSC curve of the viologen-based ionic polymer binder A5 prepared in Example 5. As shown in FIG. 5 and Table 1, the viscosity average molecular weight of the viologen-based ionic polymer binder is lower than the molecular weight of commercial PVDF. Therefore, it could be expected that under the same conditions, the slurry prepared with the viologen-based ionic polymer binder according to the present disclosure has a lower viscosity and was easier to apply. The thermal decomposition temperature of the viologen-based ionic polymer binder is slightly higher than that of PVDF (316.0° C.), such that the prepared cathode has desirable heat resistance and meets the demands of battery operation under high-temperature conditions. The glass transition temperature of the viologen-based ionic polymer binder according to the present disclosure was similar to that of PVDF (−47.0° C.) (except for the binder A5, which has a glass transition temperature of 24.5° C.), such that the prepared cathode shows high flexibility.

### Test Example 2

C1 to C10 lithium iron phosphate cathodes were assembled according to the following procedures: LiPF₆ was dissolved in a mixed solution of EC, DMC, and DEC with a volume ratio of 1:1:1 to obtain an electrolyte (in which a concentration of LiPF₆ was 1 mol/L); a polypropylene microporous membrane (Celgard 2325) was used as a separator; a lithium-ion battery was assembled with lithium metal as a counter electrode, in which, the lithium iron phosphate cathode was a cathode and the lithium metal was an anode. The stainless steel gasket, lithium metal, electrolyte, separator, electrolyte, lithium iron phosphate cathode, stainless steel gasket, and stainless steel shrapnel were placed in the center of a CR2032 anode casing in sequence, and a CR2032 cathode casing was placed on the top. The battery was placed in an MSK 110 battery packaging machine, pressurized to 50 psi in a locked state, and then unlocked to obtain a lithium iron phosphate|metal lithium battery. 40 μL of the electrolyte was used in each lithium iron phosphatellithium metal battery, and was added dropwise to both sides of the separator in an equal amount to fully wet the separator. The batteries assembled with C1 to C10 lithium iron phosphate cathodes were lithium iron phosphatellithium metal batteries, denoted as D1 to D10, respectively (the lithium iron phosphatellithium metal battery assembled with the cathode C1 prepared in Use Example 1 was denoted as D1, and so on to D10).

Battery specific capacity refers to an initial discharge specific capacity and a 400th cycle discharge specific capacity of the assembled lithium iron phosphate|metal lithium battery at a current density of 0.5C. The test instrument was a battery recycling tester (Wuhan Blue Electric Electronics Co., Ltd., China, CT3002A), with a cut-off voltage of 2.5 V to 4.2 V, a test temperature of 25° C., a rate of 0.5C, and an active material standard specific capacity of 170 mAh/g. An electrochemical workstation (VersaSTAT3, Princeton, USA) was used to test the initial interface impedance of the assembled lithium iron phosphate|metal lithium battery, during which parameters were set as follows: a test temperature of 25° C., a frequency of 1,000,000 Hz, and an amplitude of 5 mV. The density and peel strength of the lithium iron phosphate cathode, initial discharge specific capacity, initial interface impedance, 400th cycle discharge specific capacity, and capacity retention (a ratio of the discharge specific capacity to the initial discharge specific capacity) of the battery obtained by the test are shown in Table 2. The peel strength between the current collector and the dry coating on the surface of the current collector in the electrode was determined according to the test method of GB/T 2791-1995. A density of the active materials of the cathode referred to a mass of lithium iron phosphate per unit area, which was obtained by calculation.

TABLE 2
Performance indicators of lithium iron phosphate|lithium metal battery

| | Density of | Cathode | | Initial | |
|---|---|---|---|---|---|
| | lithium iron | materials | Initial | discharge | 400th cycle |
| Lithium iron | phosphate | peeling | interface | specific | discharge specific |
| phosphate\|lithium | cathode | strength | impedance | capacity | capacity (mAh · g⁻¹) |
| metal battery | (mg · cm⁻²) | (N · m⁻¹) | (Ω) | (mAh · g⁻¹) | and retention |
| D1 | 2.8 | 135.2 | 210.8 | 144.8 | 132.2 (91.30%) |
| D2 | 2.8 | 142.2 | 209.1 | 144.9 | 131.2 (90.55%) |
| D3 | 2.6 | 141.7 | 205.3 | 141.5 | 130.1 (91.94%) |
| D4 | 2.7 | 144.2 | 209.7 | 143.5 | 132.8 (92.54%) |
| D5 | 2.8 | 145.7 | 204.1 | 145.2 | 136.3 (93.87%) |
| D6 | 2.7 | 134.3 | 209.9 | 142.7 | 129.2 (90.54%) |
| D7 | 2.7 | 138.4 | 207.7 | 144.5 | 130.5 (90.31%) |
| D8 | 2.5 | 134.2 | 208.9 | 143.8 | 129.1 (89.78%) |
| D9 | 2.6 | 120.1 | 211.9 | 144.1 | 128.5 (89.17%) |
| D10 | 2.8 | <1 | 229.6 | 143.1 | 111.7 (78.06%) |

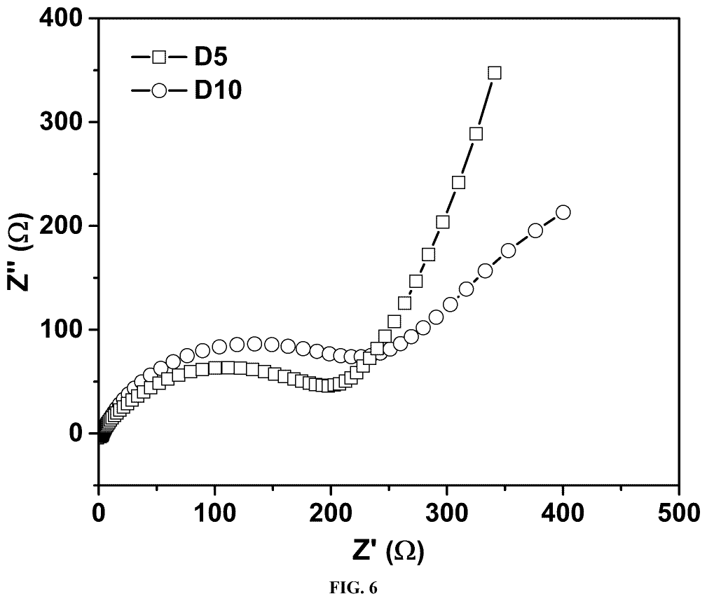

FIG. 6 shows the electrochemical impedance spectroscopy of lithium iron phosphatellithium metal batteries D5 and D10 at 25° C.

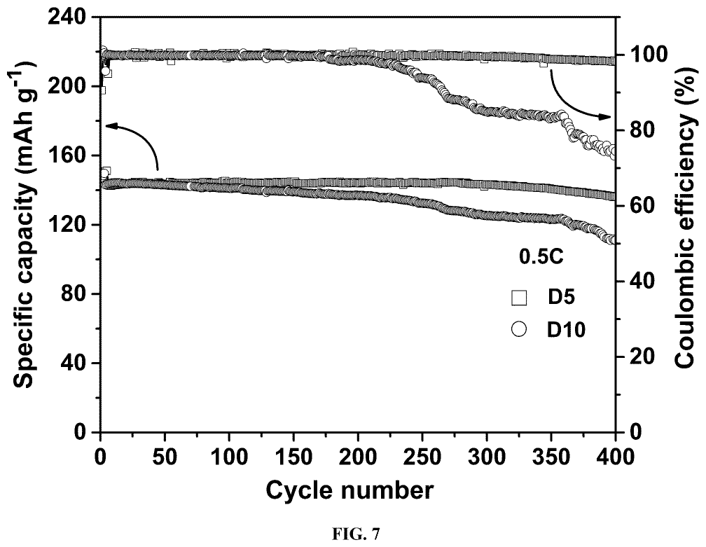

FIG. 7 shows long recycling charge-discharge curves of the lithium iron phosphatellithium metal batteries D5 and D10 at 25° C. with a cut-off voltage of 2.5 V to 4.2 V and a rate of 0.5C.

As shown in Table 2, FIG. 6, and FIG. 7, under the condition of similar density of lithium iron phosphate, the cathodes C1 to C9 have higher peel strength than C10, indicating that the viologen-based ionic copolymer binder has stronger adhesion. In addition, the lithium iron phosphate|metal lithium batteries D1 to D9 assembled using the cathodes C1 to C9 each have lower initial interface impedance than that of the lithium iron phosphate|metal lithium battery D10 assembled using the cathode C10 prepared with the PVDF binder, indicating that the ionic copolymer binder shows better lithium-ion transportation. Although the lithium iron phosphate|metal lithium batteries D1 to D9 assembled using C1 to C9 each have similar initial discharge specific capacity as that of the lithium iron phosphate|metal lithium battery D10 assembled using the cathode C10 prepared with the PVDF binder, D1 to D9 each have a higher discharge specific capacity and capacity retention after 400 cycles at a current density of 0.5C compared with D10.

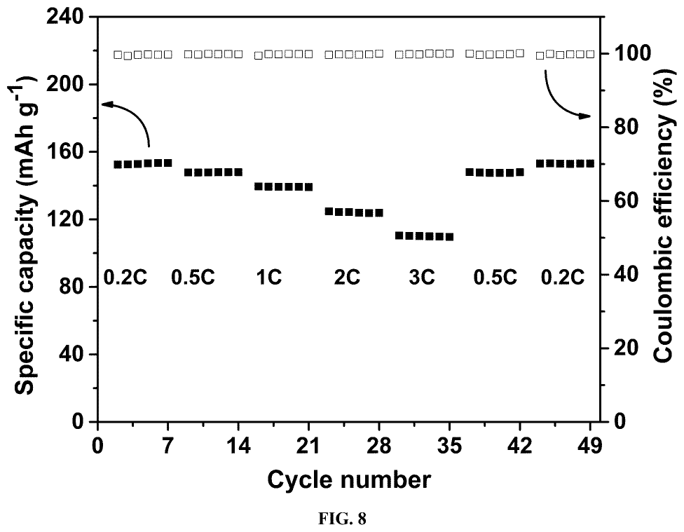

FIG. 8 shows a charge-discharge curve of the lithium iron phosphatellithium metal battery D5 at 25° C. with a cut-off voltage of 2.5 V to 4.2 V and different rates of 0.2C, 0.5C, 1C, 2C, and 3C. As shown in FIG. 8, the lithium iron phosphate|metal lithium battery could show a high discharge specific capacity at each current density, and still shows a discharge specific capacity of 110.8 mAh g⁴ even at a high current density of 3C; when the current density returns to 0.2C, the specific capacity also steadily returns to the initial value.

### Test Example 3

Characterization of parameters for lithium iron phosphate|artificial graphite full battery: the lithium iron phosphate cathode C5 provided in Use Example 5 and the lithium iron phosphate cathode C10 provided in Comparative Use Example 1 were used as the cathodes, and the artificial graphite anode C11 provided in Comparative Use Example 2 was used as the anode to assemble a lithium iron phosphate|artificial graphite full battery. The lithium iron phosphate|artificial graphite full battery assembled from the lithium iron phosphate cathode C5 provided in Use Example 5 was recorded as E1, while the lithium iron phosphate|artificial graphite full battery assembled from the lithium iron phosphate cathode C10 provided in Comparative Use Example 1 was recorded as E2.

A method for assembling a lithium iron phosphate|artificial graphite full battery was as follows: LiPF₆ was dissolved in a mixed solution of EC, DMC, and DEC with a volume ratio of 1:1:1 to obtain an electrolyte (in which a concentration of LiPF₆ was 1 mol/L); a polypropylene microporous membrane (Celgard 2325) was used as a separator; the C1 or C10 was used as the cathode; and artificial graphite electrode was used as the anode. The stainless steel gasket, artificial graphite anode, electrolyte, separator, electrolyte, lithium iron phosphate cathode, stainless steel gasket, and stainless steel shrapnel were placed in the center of a CR2032 anode casing in sequence, and a CR2032 cathode casing was placed on the top. The battery was placed in an MSK 110 battery packaging machine, pressurized to 50 psi in a locked state, and unlocked to obtain lithium iron phosphate|artificial graphite batteries E1 to E2. 40 μL of electrolyte was used in each lithium iron phosphate|artificial graphite battery, and was added dropwise to both sides of the separator in an equal amount to fully wet the separator.

The E1 to E2 lithium iron phosphate|artificial graphite batteries were subjected to battery recycling testing in a battery recycling tester (Wuhan Blue Electric Electronics Co., Ltd., China, CT3002A) with a cut-off voltage of 2.5 V to 4.2 V, a test temperature of 25° C., a rate of 0.5C, and an active material standard specific capacity of 170 mAh/g. The test results of the initial cycle specific capacity, and the 500th cycle discharge specific capacity and capacity retention (a ratio of the discharge specific capacity to the initial discharge specific capacity) of the battery are shown in Table 3.

TABLE 3
Performance indicators of lithium iron phosphate|artificial graphite battery

| Lithium iron | | |
|---|---|---|
| phosphate\|artificial | Initial discharge specific | 500th cycle discharge specific |
| graphite battery | capacity (mAh g⁻¹) | capacity (mAh · g⁻¹) and retention |
| E1 | 146.6 | 140.1 (95.56%) |
| E2 | 145.7 | 128.9 (89.89%) |

As shown in Table 3, the lithium iron phosphate|artificial graphite full battery assembled with the cathode C5 prepared with the viologen-based ionic polymer binder according to the present disclosure has higher specific capacity and capacity retention at a current density of 0.5C, compared with the lithium iron phosphate|artificial graphite full battery assembled with the cathode C10 prepared with the PVDF binder.

In summary, the present disclosure provides a viologen-based ionic polymer binder prepared from the polymerization of viologen-based acrylate, at least one of an acrylate and poly(ethylene glycol) methyl ether acrylate, and use thereof in the preparation of a cathode of a lithium-ion battery. The lithium-ion battery assembled therefrom exhibits low interface impedance, high specific capacity, high capacity retention, and excellent recycling stability.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A viologen-based ionic polymer binder, having a chemical structure shown in formula I: 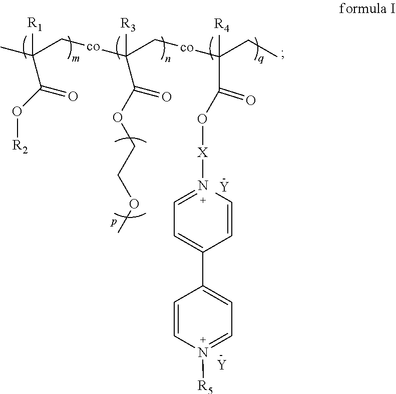
wherein in formula I, R₁, R₃, and R₄ are independently selected from the group consisting of hydrogen and methyl; R₂ and R₅ are independently alkyl; X is selected from the group consisting of methylene group, methylene ester group, and methylene carbamate group; Y is selected from the group consisting of PF₆, BF₄, Br, I, ½[SO₄], ½[CO₃], CH₃SO₃, CF₃SO₃, and (CF₃SO₂)₂N; m, n, and q satisfy m+n+q=1, where m>0, n>0, and q>0; and p is an integer in a range of 0 to 40.

2. A method for preparing the viologen-based ionic polymer binder as claimed in claim 1, comprising the steps of
mixing a viologen-based acrylate, at least one of an acrylate and poly(ethylene glycol) methyl ether acrylate, an initiator, and a first solvent, and subjecting a resulting mixture to free radical polymerization, to obtain the viologen-based ionic polymer binder; wherein
the viologen-based acrylate has a chemical structure shown in formula II: 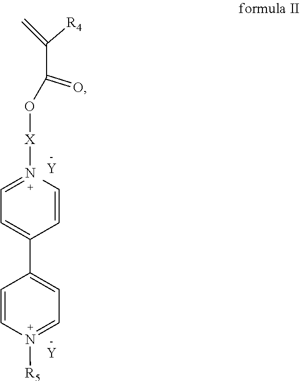
wherein in formula II, R₄ is selected from the group consisting of hydrogen and methyl; R₅ is alkyl; X is selected from the group consisting of methylene group, methylene ester group, and methylene carbamate group; and Y is selected from the group consisting of PF₆, BF₄, Br, I, ½[SO₄], ½[CO₃], CH₃SO₃, CF₃SO₃, and (CF₃SO₂)₂N.

3. The method as claimed in claim 2, wherein the viologen-based acrylate is prepared by a process comprising the steps of
(1) mixing 4,4′-bipyridine, an alkylating agent, and a second solvent, and subjecting a resulting mixture to a first alkylation reaction to obtain an N-alkyl-4,4′-bipyridine salt;
(2) mixing the N-alkyl-4,4′-bipyridine salt obtained in step (1) with a 1-halogeno alkan-1′-ol and a third solvent, and subjecting a resulting mixture to a second alkylation reaction, to obtain a 1-(hydroxyalkyl)-1′-alkyl viologen salt (1);
(3) mixing the 1-(hydroxyalkyl)-1′-alkyl viologen salt (1) obtained in step (2), a salt containing an anion different from that of the 1-(hydroxyalkyl)-1′-alkyl viologen salt (1), and a fourth solvent, and subjecting a resulting mixture to ion exchange reaction, to obtain a 1-(hydroxyalkyl)-1′-alkyl viologen salt (2); and
(4) mixing the 1-(hydroxyalkyl)-1′-alkyl viologen salt (2) obtained in step (3) with a vinyl carbonyl compound, a catalyst, a polymerization inhibitor, and a fifth solvent, and subjecting a resulting mixture to esterification reaction, to obtain the viologen-based acrylate.

4. The method as claimed in claim 3, wherein in step (1),
the alkylating agent is at least one selected from the group consisting of methyl iodide, dimethyl sulfate, dimethyl carbonate, ethyl iodide, diethyl sulfate, diethyl carbonate, propyl iodide, dipropyl sulfate, and dipropyl carbonate;
the second solvent is at least one selected from the group consisting of dichloroethane, tetrahydrofuran (THF), dichloromethane, toluene, and chloroform; and
the first alkylation reaction is conducted at a temperature of 10° C. to 35° C. for 1 h to 24 h.

5. The method as claimed in claim 3, wherein in step (2),
the 1-halogeno alkan-1′-ol is at least one selected from the group consisting of 2-bromoethanol, 2-chloroethanol, 3-bromo-1-propanol, 3-chloro-1-propanol, 4-bromo-1-butanol, 4-chloro-1-butanol, 5-bromo-1-pentanol, 5-chloro-1-pentanol, 6-bromo-1-hexanol, 6-chloro-1-hexanol, 7-bromo-1-heptanol, 7-chloro-1-heptanol, 8-bromo-1-octanol, and 8-chloro-1-octanol;
the third solvent is at least one selected from the group consisting of acetonitrile, N-methylpyrrolidone (NMP), N,N-dimethylformamide (DMF), and dimethyl sulfoxide (DMSO); and
the second alkylation reaction is conducted at a temperature of 50° C. to 100° C. for 30 h to 72 h.

6. The method as claimed in claim 3, wherein in step (3),
the salt containing the anion different from that of the 1-(hydroxyalkyl)-1′-alkyl viologen salt (1) is at least one selected from the group consisting of KPF₆, NaPF₆, NH₄PF₆, NaBF₄, KBF₄, NaBr, KBr, NaI, KI, Na₂SO₄, Na₂CO₃, K₂CO₃, CH₃SO₃Na, CF₃SO₃Na, and (CF₃SO₂)₂NLi;
the fourth solvent is at least one selected from the group of deionized water, ethanol, NMP, DMF, and DMSO; and
the ion exchange reaction is conducted at a temperature of 10° C. to 35° C. for 1 h to 12 h.

7. The method as claimed in claim 3, wherein in step (4),
the vinyl carbonyl compound is at least one selected from the group consisting of 2-isocyanatoethyl acrylate, isocyanatoethyl methacrylate, acrylic acid, methacrylic acid, acryloyl chloride, and methacryloyl chloride;
the catalyst is at least one selected from the group consisting of dibutyltin dilaurate, triethylamine, sodium carbonate, and potassium carbonate;
the polymerization inhibitor is at least one selected from the group consisting of p-methoxyphenol, p-benzoquinone, and p-hydroquinone;
the fifth solvent is at least one selected from the group consisting of NMP, DMF, THF, 1,4-dioxane, acetonitrile, and chloroform; and
the esterification reaction is conducted at a temperature of 10° C. to 35° C. for 1 h to 36 h.

8. The method as claimed in claim 2, wherein the initiator for the free radical polymerization is at least one selected from the group consisting of azobisisobutyronitrile, 1,1′-azobis(cyclohexane-1-carbonitrile), dimethyl 2,2′-azobisisobutyrate, and dibenzoyl peroxide;
the first solvent is at least one selected from the group consisting of acetonitrile, toluene, acetone, NMP, DMF, THF, 1,4-dioxane, and chloroform; and
the free radical polymerization is conducted at a temperature of 50° C. to 80° C. for 8 h to 12 h.

9. A method for preparing an electrode of a lithium-ion battery, comprising
1) dissolving the viologen-based ionic polymer binder as claimed in claim 1 in a first organic solvent to obtain a binder solution;
2) mixing an electrode active material and a conductive material to obtain a mixed powder;
3) mixing the binder solution obtained in step 1), the mixed powder obtained in step 2), and a second organic solvent to obtain an electrode slurry; and
4) applying the electrode slurry obtained in step 3) onto a current collector to obtain the electrode of the lithium-ion battery.

10. The method as claimed in claim 9, wherein the electrode of the lithium-ion battery is one selected from the group consisting of a cathode and an anode.
